(19) United States
(12) Patent Application Publication
(10) Pub. No.: US 2024/0271447 A1
(43) Pub. Date: Aug. 15, 2024

(54) EQUIPMENT FOR SWIMMING POOL OR SPA WITH CONTACTLESS WATER DETECTION

(71) Applicant: ZODIAC POOL CARE EUROPE, Belberaud (FR)

(72) Inventors: Arnaud Besnier, Beaupuy (FR); Mehdi Chafik, Tournefeuille (FR)

(21) Appl. No.: 18/439,383

(22) Filed: Feb. 12, 2024

Related U.S. Application Data
(60) Provisional application No. 63/445,167, filed on Feb. 13, 2023.

(51) Int. Cl. E04H 4/16; G01N 33/18

(52) U.S. Cl. CPC E04H 4/1654; G01N 33/18

(57) ABSTRACT

Systems and methods for operating equipment for a swimming pool or spa include detecting water without contacting water of the swimming pool or spa. The systems and methods may be utilized with various types of equipment for a swimming pool or spa, such as but not limited to a cleaner, an automatic swimming pool cleaner, a skimmer, a docking station, a heater, and/or a pump. The systems and methods include one or more sensors detecting water without contact the water.

### CROSS REFERENCE TO RELATED APPLICATION

This application claims the benefit of U.S. Provisional Patent Application No. 63/445,167, filed on Feb. 13, 2023, and entitled EQUIPMENT FOR SWIMMING POOL OR SPA WITH CONTACTLESS WATER DETECTION, the content of which is hereby incorporated by reference in its entirety.

### FIELD OF THE INVENTION

The invention relates to systems and apparatuses for water-containing vessels such as swimming pools and spas and more particularly, although not necessarily exclusively, to equipment such as automatic swimming pool cleaners (APCs), pumps, and/or heaters with water detection.

### BACKGROUND OF THE INVENTION

Swimming pools and spas often include equipment that perform various actions relating to the water of the swimming pools or spas. As examples, a swimming pool or spa system may include robotic cleaners such as but not limited to automatic swimming pool cleaners (APCs), pumps for circulating the water, heaters for heating the water, etc. Some equipment for swimming pools or spas may include water sensors that detect water based on contact of water with the sensors, and based on such detected contact (or lack of contact), the equipment may perform various operations. However, traditional contact water sensors are susceptible to corrosion and provide a location on the equipment where waterproofness may be compromised and/or the risk of water leakage into the equipment is increased.

### SUMMARY

Embodiments covered by this patent are defined by the claims below, not this summary. This summary is a high-level overview of various embodiments and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification of this patent, any or all drawings, and each claim.

According to certain embodiments, a piece of equipment for a swimming pool or spa includes a sensor which may detect water without contacting water.

According to various embodiments, a piece of equipment for a swimming pool or spa includes a contactless water detection sensor.

According to some embodiments, a method of operating equipment for a swimming pool or spa includes detecting a presence of water with a contactless water sensor.

According to various embodiments, a method of operating equipment for a swimming pool or spa includes detecting a presence of water or an absence of water with a contactless water sensor. In various embodiments, the method includes generating an output based on the detected presence or absence of water.

According to certain embodiments, a piece of equipment for a swimming pool or spa includes a wall and a contactless water sensor. In various embodiments, the wall includes a first side at least partially defining a dry area of the piece of equipment and a second side configured to be exposed to water. In certain embodiments, the contactless water sensor may be within the dry area.

Various implementations described herein can include additional systems, methods, features, and advantages, which cannot necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

### BRIEF DESCRIPTION OF THE DRAWINGS

The specification makes reference to the following appended figures, in which use of like reference numerals in different figures is intended to illustrate like or analogous components.

FIG. 1 illustrates a pool system according to embodiments.

FIG. 2 illustrates the pool system of FIG. 1 according to embodiments.

### DESCRIPTION OF THE INVENTION

Described herein are systems and methods for detecting water without contacting water of a swimming pool or spa. The systems and methods described herein may be utilized with various types of equipment for a swimming pool or spa. As non-limiting examples, the systems or methods described herein may be used with a cleaner such as an APC, a skimmer, a docking station, a pump, a heater, combinations thereof, and/or other equipment for a swimming pool or spa as desired. Compared to traditional approaches in which water sensors of pool equipment require direct contact between the water sensor and the water of the pool or spa, the systems and methods described herein include one or more sensors detecting water without contacting the water, and thus may be considered contactless sensors. In various embodiments, the sensors described herein may be conductive-or resistive-based sensors. Additionally, or alternatively, the sensors described herein may be capacitive-based sensors. A plurality of contactless sensors may be used on a given piece of equipment in certain embodiments. In some embodiments, the sensors described herein may be provided within a dry area of the piece of equipment. The systems and methods described herein may minimize or prevent corrosion of the sensor and may reduce the number of potential leak locations on the piece of equipment. Various other benefits and advantages may be realized with the systems and methods provided herein, and the aforementioned advantages should not be considered limiting.

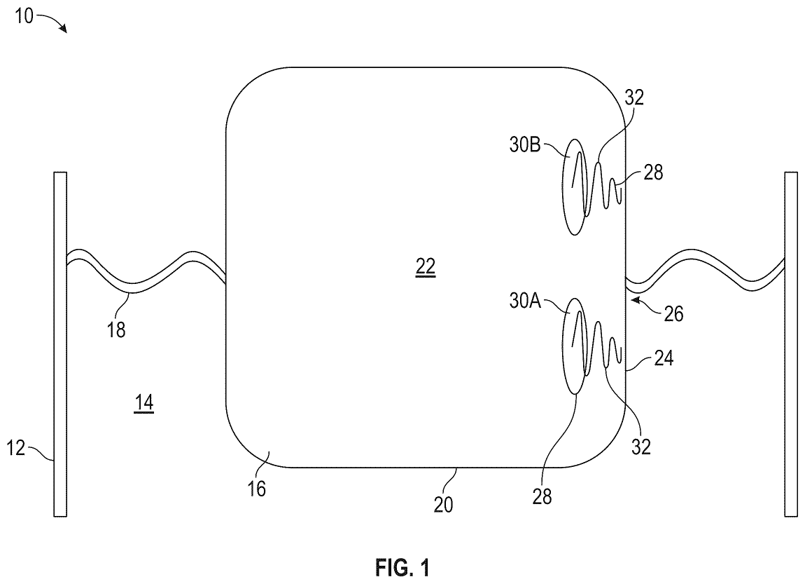

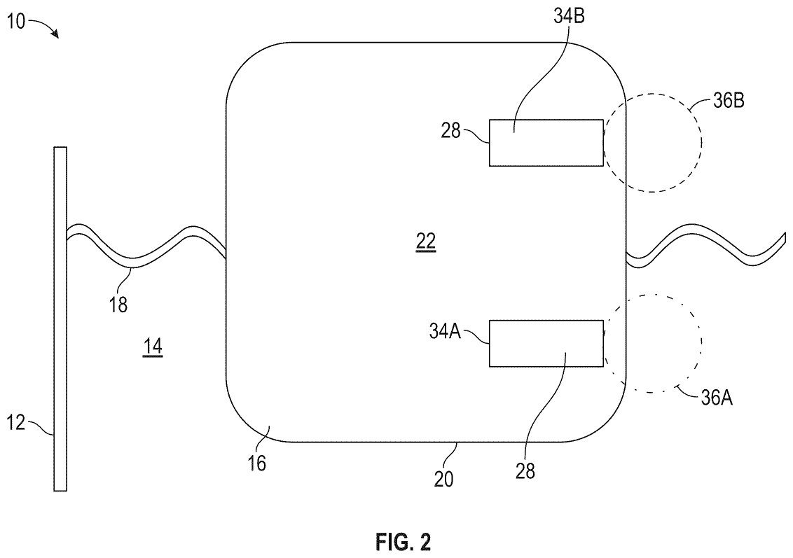

FIGS. 1 and 2 illustrate an example of a pool system 10 that generally includes a pool or spa 12 with a volume of water 14 and a piece of equipment 16. The piece of equipment 14 may be various types of equipment as desired and may be adapted to float at a waterline 18 of the pool or spa 12, be submerged within the water 14, and/or otherwise operate in and/or around the water 14 as desired. In some embodiments, the equipment need not be submergible or positioned within the water 14 of the pool or spa 12, and the equipment 16 instead may be associated with other equipment positionable within the water 14. A non-limiting example of such equipment may be a docking station for an APC. Moreover, in certain embodiments, the piece of equipment need not be movable relative to the water 12 and instead may be relatively fixed (e.g., such as a heater or a pump, among other equipment). Other non-limiting examples of equipment 16 may be cleaners such as but not limited to APCs, a docking station for the APC, skimmers, filter systems, combinations thereof, and/or other types of equipment as desired. As a non-limiting example, the equipment may be an APC such as those described in U.S. Pat. Nos. 10,316,534, 9,488,154, 8,578,538, and U.S. Patent Publication No. 2014/0303810, all of which are hereby incorporated by reference in their entireties. While a single piece of equipment is illustrated, any number of pieces of equipment 16 may be utilized as desired.

As illustrated in FIGS. 1 and 2, regardless of the type of equipment 16, the equipment 16 generally includes a housing or compartment 20 defining one or more dry areas 22. In one non-limiting example, the dry area 22 may be an electronics compartment of the equipment 16, although it need not be in other examples. As illustrated in FIG. 1, at least one wall 24 may at least partially define the dry area 22 while having an external side or surface 26 opposite from the dry area 22 that may be exposed to water (e.g., water 14 of the pool or spa 12, rain, etc.).

In various embodiments, the equipment 16 includes one or more contactless water sensors 28 within the dry area 22 and adjacent to the wall 24. The contactless water sensors 28 may detect a presence or absence of water without contacting the water. Non-limiting examples of contactless water sensors 28 may be capacitive sensors, resistive sensors, and/or conductive sensors, among others. In these embodiments, the contactless water sensors 28 may detect water based on a change in conductivity, a change in capacity, and/or a change in resistivity due to the presence of water but without contacting the water. In the embodiment illustrated, the equipment 16 includes conductive or resistive buttons 30A-B that are supported on springs 32. In this example, contact and/or immersion of the surface 26 with water causes a change in resistance or conductivity, which in turn may cause the conductive or resistive buttons to compress the springs 32 (e.g., towards the wall 24). Such “pressing” of the conductive or resistive buttons may provide a signal to the equipment 16 that water is detected and/or the equipment 16 is immersed. Conversely, when water is not present on the wall 24, the conductive or resistive buttons 30A-B are not compressed, and the “signal” is lost, thereby indicating that the equipment 16 is no longer immersed and/or water is no longer detected.

In another non-limiting example, submerging and/or water on the surface 26 may create a link or signal between the conductive or resistive buttons 30A-B, thereby allowing the equipment 16 to sense that the equipment 16 is in water or exposed to water (e.g., rain).

Referring to FIG. 2, in a further non-limiting example, the contactless water sensors 28 may be capacitive sensors 34A-B within the dry area 22 and adjacent to the wall 24. In such embodiments, the capacitive sensors 34A-B may emit an electrical field (represented by dashed circles 36A-B) from a sensing portion of the capacitive sensors 34A-B. A change in the electrical fields 36A-B (e.g., due to the presence or absence of water) may be used to allow the equipment 16 to sense that the equipment 16 is in or exposed to water. In FIG. 2, the change in electrical fields detected by the capacitive sensors 34A-B is represented by the different pattern of dashes of electrical filed 36A compared to electrical field 36B.

The contactless water sensors 28 of FIG. 1 are for illustrative purposes only, and other arrangements and/or types of contactless water sensors 28 may be utilized with the equipment 16.

Optionally, the equipment 16 may generate an output based on the detection (or lack of detection) of water by the contactless water sensors 28. As a non-limiting example, a contactless water sensor 28 may be on an APC or on a docking station for an APC, and the contactless water sensor 28 may detect rainfall on the APC and/the docking station. In such embodiments, the APC may stop a cleaning cycle and/or the docking station may stop charging of the APC and/or may generate an alert to a user based on the detection of the rainfall. As a further non-limiting example, a contactless water sensor 28 may be on an APC, and the APC may initiate a cleaning cycle based on a detected presence of water from the contactless water sensor 28 and/or send a notification to a user and/or display a notification and/or stop a cleaning cycle based on a detected absence of water. Various other outputs may be implemented as desired.

Exemplary concepts or combinations of features of the invention may include:
A. A piece of equipment for a swimming pool or spa, the piece of equipment comprising a sensor configured to detect water without contacting water.
B. The piece of equipment of any preceding or subsequent statement or combination of statements, wherein the piece of equipment comprises an APC or a docking station for the APC.
C. The piece of equipment of any preceding or subsequent statement or combination of statements, wherein the sensor comprises a conductivity or resistivity sensor.
D. The piece of equipment of any preceding or subsequent statement or combination of statements, wherein the sensor comprises a capacitive sensor.
E. The piece of equipment of any preceding or subsequent statement or combination of statements, wherein the sensor comprises a pair of resistive buttons.
F. The piece of equipment of any preceding or subsequent statement or combination of statements, wherein the piece of equipment comprises a wall at least partially defining a dry area, wherein a surface of the wall opposite from the dry area is configured to be exposed to water, and wherein the sensor is within the dry area.
G. A piece of equipment for a swimming pool or spa, the piece of equipment comprising a contactless water detection sensor.
H. The piece of equipment according to statement G, further according to any preceding or subsequent statement or combination of statements.
I. A method of operating equipment for a swimming pool or spa, the method comprising detecting a presence of water with a contactless water sensor.
J. A method of operating equipment for a swimming pool or spa, the method comprising detecting a presence or absence of water with a contactless water sensor, and generating an output based on the detected presence or absence of water.
K. The method of any preceding or subsequent statement or combination of statements, wherein generating the output comprises causing an APC to initiate a cleaning cycle based on the detected presence of water or to cease charging of the APC.
L. A piece of equipment for a swimming pool or spa, the piece of equipment comprising a wall and a contactless water sensor, wherein the wall comprises a first side at least partially defining a dry area of the piece of equipment and a second side configured to be exposed to water, wherein the contactless water sensor is within the dry area.
M. The piece of equipment according to statement L, further according to any preceding or subsequent statement or combination of statements.

These examples are not intended to be mutually exclusive, exhaustive, or restrictive in any way, and the invention is not limited to these example embodiments but rather encompasses all possible modifications and variations within the scope of any claims ultimately drafted and issued in connection with the invention (and their equivalents). For avoidance of doubt, any combination of features not physically impossible or expressly identified as non-combinable herein may be within the scope of the invention. Further, although applicant has described devices and techniques for use principally with APCs, persons skilled in the relevant field will recognize that the present invention conceivably could be employed in connection with other objects and in other manners. Finally, references to “pools” and “swimming pools” herein may also refer to spas or other water containing vessels used for recreation, training, or therapy and for which cleaning of debris is needed or desired.

That which is claimed:

1. An automatic pool cleaner (APC) for a swimming pool or spa or a docking station for the APC, the APC and/or the docking station comprising a sensor configured to detect water without contacting water.

2. The APC or docking station of claim 1, wherein the sensor comprises a conductivity or resistivity sensor.

3. The APC or docking station of claim 1, wherein the sensor comprises a capacitive sensor.

4. The APC or docking station of claim 1, wherein the sensor comprises a pair of resistive buttons.

5. The APC or docking station of claim 1, wherein the piece of equipment comprises a wall at least partially defining a dry area, wherein a surface of the wall opposite from the dry area is configured to be exposed to water, and wherein the sensor is within the dry area.

6. A method of operating an automatic pool cleaner (APC) for a swimming pool or spa or a docking station for the APC, the method comprising detecting a presence or absence of water on at least one of the APC or the docking station with a contactless water sensor, and generating an output based on the detected presence or absence of water.

7. The method of claim 6, wherein generating the output comprises causing the APC to initiate a cleaning cycle based on the detected presence of water on the APC.

8. The method of claim 7, wherein generating the output comprises causing the APC to cease the cleaning cycle of the APC based on the detected absence of water.

9. The method of claim 6, wherein generating the output comprises causing the APC to cease charging of the APC based on the detected presence of water.

10. An automatic pool cleaner (APC) for a swimming pool or spa or a docking station for the APC, the APC or the docking station comprising a wall and a contactless water sensor, wherein the wall comprises a first side at least partially defining a dry area of the piece of equipment and a second side configured to be exposed to water, wherein the contactless water sensor is within the dry area.

11. The piece of equipment of claim 10, wherein the APC is configured to cease charging of the APC based on the detected presence of water.

12. The piece of equipment of claim 10, wherein the APC is configured to initiate a cleaning cycle based on the detected presence of water.

13. The piece of equipment of claim 10, wherein the docking station is configured to cease charging of the APC based on the detected presence of water.

14. The piece of equipment of claim 10, wherein the sensor comprises at least one of a conductivity or resistivity sensor, a capacitive sensor, or a pair of resistive buttons.
